(19) United States
(12) Patent Application Publication
(10) Pub. No.: US 2026/0283867 A1
(43) Pub. Date: Sep. 24, 2026

(54) RETAINING DEVICE COMPRISING TWO MATERIALS

(71) Applicant: APLIX, Le Cellier (FR)

(72) Inventor: Laurent Jacques Roland GOUJON, Le Cellier (FR)

(73) Assignee: APLIX, Le Cellier (FR)

(21) Appl. No.: 19/474,561

(22) PCT Filed: Apr. 9, 2024
(86) PCT No.: PCT/FR2024/050469

§ 371 Date: Oct. 10, 2025

(30) Foreign Application Priority Data
Apr. 14, 2023 (FR) 2303765

(51) Int. Cl. A61F 13/62

(52) U.S. Cl. CPC A61F 13/625

(57) ABSTRACT

A retaining device including a base and a plurality of retaining elements, the retaining device including a first thermoplastic material whose flexural modulus is between 200 and 4500 MPa and a second material selected from the group consisting of a second thermoplastic material whose flexural modulus is different by at least 10% and/or at least 100 MPa from the flexural modulus of the first thermoplastic material, a non-thermoplastic material present at a content strictly greater than 1% by mass of the total composition of the retaining device, a mineral and/or vegetable filler whose content is less than or equal to 30% by mass of the total composition of the retaining device, or a combination thereof.

### TECHNICAL FIELD

The present disclosure relates to the field of retaining devices, more particularly to retaining devices comprising a plurality of retaining elements, for example intended to cooperate with a hook and/or loop counterpart.

### PRIOR ART

Particularly in the field of hygiene use is made of retaining devices, for example hooks which cooperate with a loop counterpart forming an application area of the retaining device.

A disposable diaper is generally composed of an absorbent central part comprising at each end a front part forming a belt provided with two front ears and a rear part forming a belt provided with two rear ears allowing the diaper to be fixed on the person wearing the disposable diaper. Each rear ear is generally provided with a retention device, for example with hooks, which cooperates with an application area disposed on the front belt. This application area is commonly called in the field of hygiene “comfort strip” or identified by the expression “landing zone”.

In France, 3.5 billion disposable diapers are consumed each year.

Disposable diapers take hundreds of years to degrade naturally in landfills.

Incineration is an inefficient method given the amount of liquid that must be evaporated.

Recycling can be a viable solution for conserving materials extracted from the environment, but recycling is also complex. Used disposable diapers must be cleaned of urine and feces, then the various materials they are made of must be separated and sorted.

Composting may be a possible solution. Indeed, a used disposable diaper contains approximately 75% biodegradable materials (urine, fecal matter, cellulose). However, the retaining devices, front strip, and chassis of the disposable diapers are generally made of polypropylene, which is not compostable.

Furthermore, global shortages of raw materials have resulted in the rationalization of raw material production and therefore the scarcity of very specific materials in many areas, particularly in the production of small products of the order of a few millimeters or less, for example hooks for retaining devices.

The continued need for retaining devices and the rationalization of materials available on the market have therefore introduced a need to find solutions to continue manufacturing retaining devices without introducing considerable cost increases while taking into account the considerable increases in the cost of energy and raw materials as well as existing manufacturing methods.

In addition, the materials available, particularly on the market, increasingly come from external or internal recycling channels, for example pre-consumer and/or post-consumer materials, which are in a degraded, or even very degraded, state, not allowing them to be used directly in the manufacture of retaining devices. Since such materials are available, there is also a need to upgrade these materials.

### DISCLOSURE OF THE INVENTION

This presentation aims at overcoming at least part of these disadvantages.

For this purpose, this disclosure relates to a retaining device, comprising:
a base extending in a longitudinal direction having an upper face and a lower face,
a plurality of retaining elements extending from the upper face of the base, each retaining element comprising a rod,
the retaining device comprising predominantly, in mass percentage, in particular consisting in that it is formed from a first thermoplastic material having a flexural modulus, preferably measured according to standard ISO 178:2019, greater than or equal to 200 MPa and less than or equal to 4500 MPa and a second material, the second material being chosen from the group consisting of:
a second thermoplastic material having a flexural modulus different by at least 10% and/or at least 100 MPa from the flexural modulus of the first thermoplastic material,
a non-thermoplastic material present at a content strictly greater than 1% by mass of the total composition of the retaining device, in particular greater than or equal to 1.1%, in particular greater than or equal to 1.2%,
a mineral and/or vegetable filler whose content is less than or equal to 30% by mass of the total composition of the retaining device, or
a combination thereof.

Thanks to the composition of the retaining device, the retaining device can be compostable in an industrial environment, for example according to standard EN 13432:2000, or perhaps based on compostable materials, and/or derived from biomass, for example according to standard EN 16785-1:2016 and/or standard ASTM D 6866:2022, and can be taken care of at the end of its life in industrial composting and/or recycling channels.

Due to the composition of the retaining device, the retaining device can be made from recycled material, for example pre-consumer and/or post-consumer material.

Thanks to the composition of the retaining device, it is possible to continue the manufacture of retaining elements without radically changing the current manufacturing lines of such retaining devices and/or the methods for manufacturing such retaining devices and/or to obtain retaining devices with performances at least equivalent to those of the prior art or even better.

Thanks to the composition of the retaining device, surprisingly, it is possible to obtain retaining devices and/or preforms intended to form retaining devices which are easier to shape, in particular to mold and/or to demold and/or to calender while having a shaping temperature range acceptable for current manufacturing lines and/or for current methods for manufacturing such retaining devices.

It is understood by majority that the mass percentage of the first thermoplastic material is greater than the mass percentages of each material constituting the second material.

By way of non-limiting examples, the mass percentage of the first thermoplastic material may be greater than or equal to 30%, 40%, 50%, 60%, 70%, 80%, 90%, 95% of the mass percentage of the retaining device.

A thermoplastic material is a material capable of being repeatedly softened by heating, hardened by cooling within a temperature range characteristic of the plastic material in question and, in the softened state, of being shaped by plasticity in a mold, by extrusion or by forming, according to section 2.1177 of standard EN ISO 472:2013.

It is understood that the first thermoplastic material and/or the second thermoplastic material are materials that have thermoplastic behavior. However, these thermoplastic materials may comprise non-thermoplastic materials in such an amount that the behavior of the material is still a thermoplastic behavior.

A mineral and/or vegetable filler is a relatively inert solid material, added in particular to modify the resistance of the material, in particular the shaping of the material and/or the stability of the material and/or the processing properties, for example shaping, of the material and/or reduce production costs.

By way of non-limiting examples, the filler may be in the form of powder or fibers.

The content of the mineral and/or vegetable filler may be greater than or equal to 0.5% by mass of the total composition of the retaining device, in particular greater than or equal to 1%, 2%, 5% by mass of the total composition of the retaining device and/or less than or equal to 25% by mass of the total composition of the retaining device, in particular less than or equal to 20%.

In some embodiments, the first thermoplastic material may have a flexural modulus greater than or equal to 800 MPa, in particular greater than 1200 MPa, in particular greater than 1800 MPa, and/or less than or equal to 4000 MPa, in particular less than 3000 MPa, in particular less than 2400 MPa. Thus, the effect of the present invention, in particular recycling, can be expressed more effectively in these ranges.

In some embodiments, the first thermoplastic material may have a flexural modulus greater than or equal to 800 MPa, in particular greater than 1000 MPa, in particular greater than 1200 MPa, and/or less than or equal to 4000 MPa, in particular less than 3000 MPa, in particular less than 2400 MPa. Thus, the effect of the present invention, in particular recycling, can be expressed more effectively in these ranges.

In some embodiments, the second thermoplastic material may have a flexural modulus greater than or equal to 800 MPa, in particular greater than 1200 MPa, in particular greater than 1500 MPa, and/or less than or equal to 4000 MPa, in particular less than 3000 MPa, in particular less than 2500 MPa, more particularly less than 2000 MPa. Thus, the effect of the present invention, in particular recycling, can be expressed more effectively in these ranges and the gripping performance with a loop counterpart is improved.

In some embodiments, the flexural modulus of the first thermoplastic material may be greater than the flexural modulus of the second thermoplastic material. Thus, the effect of the present invention can be expressed more effectively in these ranges and the gripping performance with a loop counterpart is improved. In some embodiments, the flexural modulus of the first thermoplastic material may be less than the flexural modulus of the second thermoplastic material. Thus, the resulting retaining device has the advantage of being even more economical.

In some embodiments, the first thermoplastic material may have a number-average molecular mass greater than or equal to 20 000 g/mol.

The number-average molecular mass can be measured by high-temperature SEC (as in the acronym for “Size Exclusion Chromatography”).

In some embodiments, the non-thermoplastic material may comprise a plasticizer and/or a flow enhancer and/or a lubricant and/or a stabilizer.

In some embodiments, the non-thermoplastic material may comprise a molding agent and/or a release agent and/or a processing aid.

A plasticizer is a compound that lowers the softening range of a material, making it easier to shape.

A flow enhancer is a compound that reduces the viscosity of a material.

A lubricant is a compound that reduces the friction of the material on itself or on surfaces that the material contacts during its processing, for example its shaping.

A stabilizer is a compound that increases the stability of the material over time and/or in various environments.

In some embodiments, the non-thermoplastic material may simultaneously have several effects, for example that of a plasticizer and/or a flow enhancer and/or a lubricant and/or a processing aid.

In some embodiments, the second material may comprise a non-thermoplastic material having a molecular mass at least 10% lower than the number-average molecular mass of the first thermoplastic material and/or less than or equal to 15 000 g/mol.

In some embodiments, the second thermoplastic material can have several effects simultaneously, for example that of plasticizer and/or flow enhancer and/or lubricant and/or processing aid.

In some embodiments, the first thermoplastic material and/or the second thermoplastic material may have an MFR greater than or equal to 1 g/10 min, for example greater than or equal to 2 g/10 min, in particular greater than or equal to 10 g/10 min, in particular greater than or equal to 15 g/10 min and/or less than or equal to 300 g/10 min, in particular less than or equal to 200 g/10 min, in particular less than or equal to 170 g/10 min, more particularly less than 100 g/10 min, even more particularly less than 70 g/10 min. Thus, the effect of the present invention can be expressed more effectively in these ranges and the material is sufficiently fluid to form retaining elements according to the invention (that is to say enter the molding cavities) and not simply and only spread on the surface of the molding tool (outside the molding cavity), in particular for the production of retaining elements having heights of less than 350 microns and/or a base having a thickness of less than 120 micrometers, in particular less than 100 micrometers.

MFR is the mass melt flow rate of thermoplastic materials and is measured according to ISO 1133-1:2022 and/or ISO 1133-2:2011, for example at 230° C./2.16 kg or at 190° C./2.16 kg depending on their usual forming temperature of the material to be considered.

In some embodiments, the second material, in particular the second thermoplastic material, may have an MFR different by at least 10%, in particular at least 15% and/or at least 5 g/10 min, in particular at least 10 g/10 min from the MFR of the first thermoplastic material.

In some embodiments, the second material may comprise the non-thermoplastic material, the non-thermoplastic material being, at a temperature less than or equal to 150° C., in particular less than or equal to 100° C., in particular at room temperature (of the order of 25° C.), in the form of a liquid or in an intermediate form between a liquid form and a solid form, in particular in the form of a wax.

In some embodiments, the non-thermoplastic material may have a kinematic viscosity of less than or equal to 700 mm²/s, measured according to ASTM D445-21E02, at the processing temperature, for example at the forming temperature.

The processing temperature, in particular the shaping temperature, may be greater than or equal to 80° C., in particular greater than or equal to 95° C. and/or less than or equal to 250° C., in particular less than or equal to 225° C., in particular less than or equal to 200° C.

In some embodiments, the non-thermoplastic material may have a dynamic viscosity of less than or equal to 500 mPas·s, measured according to ASTM D445-21E02, at the processing temperature, for example at the forming temperature.

The processing temperature, in particular the shaping temperature, may be greater than or equal to 80° C., in particular greater than or equal to 95° C. and/or less than or equal to 250° C., in particular less than or equal to 225° C., in particular less than or equal to 200° C.

By way of non-limiting example, the first and/or second thermoplastic material comprises:
a polyolefin, in particular a polypropylene (PP) and/or a polyethylene (PE), in particular a bio-sourced polyethylene (Bio-PE) and/or their copolymers, and/or
a polyester, in particular a polyethylene terephthalate (PET), for example a bio-sourced polyethylene terephthalate (Bio-PET), and/or a biodegradable polyester and/or a Polyhydroxyalkanoate (PHA) and/or a poly-lactic acid (PLA) and/or a polybutylene adipate terephthalate (PBAT) and/or a Polybutylene succinate (PBS) and/or a poly(butylene succinate co-butylene adipate) (PBSA) and/or a Poly(Polybutylene succinate) (PES) and/or a poly(trimethylene succinate) (PTS) and/or a poly(tetramethylene adipate-co-terephthalate) (PTAT) and/or a Polycaprolactone (PCL) and/or a Thermoplastic starch (TPS), and/or
a polyamide (PA) in particular a bio-sourced polyamide (Bio-PA), and/or a casein derivative and/or
a mixture of two or more of these thermoplastic materials.

In some embodiments, the retaining device may be free of a colorant, for example a TiO₂-based colorant.

In some embodiments, the second material may comprise a mineral and/or vegetable filler, for example a colorant, for example a TiO₂-based colorant, in particular at a mass percentage of the composition less than or equal to 1.5%, in particular less than or equal to 1%.

In some embodiments, the first thermoplastic material may be a first polyolefin-based material, for example, a first material based on a first polypropylene, and the second material may comprise the second thermoplastic material, the second thermoplastic material being a second polyolefin-based material, for example, a second polypropylene.

In some embodiments, the ratio of the mass percentage of the second thermoplastic material to the first thermoplastic material may be greater than or equal to 0.3, in particular greater than or equal to 0.45, and/or less than or equal to 0.7, in particular less than or equal to 0.60.

In some embodiments, the MFR of the first thermoplastic material may be greater than or equal to 10 g/10 min, in particular greater than or equal to 15 g/10 min, and/or less than or equal to 30 g/10 min, in particular less than or equal to 25 g/10 min.

In some embodiments, the MFR of the first thermoplastic material may be greater than or equal to 25 g/10 min, in particular greater than or equal to 35 g/10 min, and/or less than or equal to 45 g/10 min, in particular less than or equal to 40 g/10 min.

In some embodiments, the MFR of the second thermoplastic material may be greater than or equal to 50 g/10 min, in particular greater than or equal to 55 g/10 min, and/or less than or equal to 70 g/10 min, in particular less than or equal to 65 g/10 min.

In some embodiments, the MFR of the second material thermoplastic may be greater than or equal to 10 g/10 min, in particular greater than or equal to 15 g/10 min, and/or less than or equal to 40 g/10 min, in particular less than or equal to 35 g/10 min.

In some embodiments, the flexural modulus of the first thermoplastic material may be greater than or equal to 1900 MPa, in particular greater than or equal to 2000 MPa, and/or less than or equal to or equal to 2500 MPa, in particular less than or equal to 2300 MPa.

In some embodiments, the flexural modulus of the first thermoplastic material is greater than or equal to 1300 MPa, in particular greater than or equal to 1500 MPa, and/or less than or equal to 2000 MPa, in particular less than or equal to 1900 MPa.

In some embodiments, the flexural modulus of the second thermoplastic material may be greater than or equal to 1500 MPa, in particular greater than or equal to 1700 MPa, and/or less than or equal to 2400 MPa, in particular less than or equal to 2000 MPa, for example strictly less than or equal to 2000 MPa.

In some embodiments, the flexural modulus of the second thermoplastic material may be greater than or equal to 1100 MPa, in particular greater than or equal to 1300 MPa, and/or less than or equal to 1800 MPa, in particular less than or equal to 1700 MPa.

In some embodiments, the first thermoplastic material may be based on a polyester, and the second material may comprise a non-thermoplastic material present at a content greater than or equal to 1.5% by mass of the total composition of the retaining device, in particular greater than or equal to 2%, in particular greater than or equal to 5%, and/or less than or equal to 25% by mass of the total composition of the retaining device, in particular less than or equal to 20%, in particular less than or equal to 18%, more precisely less than or equal to 15%, in certain cases less than or equal to 13%.

In some embodiments, the MFR of the first polyester-based thermoplastic material may be greater than or equal to 10 g/10 min, in particular greater than or equal to 20 g/10 min, and/or less than or equal to 50 g/10 min, in particular less than or equal to 40 g/10 min.

In some embodiments, the non-thermoplastic material may have a molecular mass greater than or equal to 250 g/mol, in particular greater than or equal to 300 g/mol and/or less than or equal to 19 000 g/mol, in particular less than or equal to 15 000 g/mol, in particular less than or equal to 10 000 g/mol, more particularly less than or equal to 800 g/mol and in certain cases less than or equal to 550 g/mol.

In some embodiments, the ratio of the mass percentage of the second material to the first thermoplastic material may be greater than or equal to 0.02, in particular greater than or equal to 0.05, in particular greater than or equal to 0.1 and/or less than or equal to 0.30, in particular less than or equal to 0.25, in particular less than or equal to 0.24.

In some embodiments, the non-thermoplastic material may be based on fatty acid derivatives, for example a fatty ester and/or a fatty amide, and/or citrate and/or polyalkylene glycol and/or an azelaic acid, especially being in an oil and/or wax and/or flake form.

In some embodiments, the first thermoplastic material may be based on a first polyester, and the second material may comprise the second thermoplastic material, the second thermoplastic material being based on a second polyester.

In some embodiments, the MFR of the first thermoplastic material may be greater than or equal to 15 g/10 min, in particular greater than or equal to 25 g/10 min, and/or less than or equal to 55 g/10 min, in particular less than or equal to 45 g/10 min.

In some embodiments, the MFR of the second thermoplastic material may be less than or equal to 25 g/10 min, in particular less than or equal to 15 g/10 min, in particular less than or equal to 10 g/10 min and/or greater than or equal to 0.5 g/10 min.

In some embodiments, the flexural modulus of the first thermoplastic material may be greater than or equal to 1500 MPa, in particular greater than or equal to 2500 MPa, in particular greater than or equal to 3000 MPa and/or less than or equal to 4500 MPa, in particular less than or equal to 4000 MPa.

In some embodiments, the tensile modulus of the second thermoplastic material may be less than or equal to 2500 MPa, in particular less than or equal to 1500 MPa, in particular less than or equal to 1200 MPa, in some cases less than or equal to 800 MPa and/or greater than or equal to 15 MPa, in particular greater than or equal to 50 MPa, in particular greater than or equal to 100 MPa.

The tensile modulus is measured according to ISO 527-1:2019 and/or ISO 527-2:2012.

In some embodiments, the ratio of the mass percentage of the second thermoplastic material, for example of the second polyester of the second thermoplastic material, to the first thermoplastic material, for example of the first polyester of the first thermoplastic material, may be greater than or equal to 0.20, in particular greater than or equal to 0.30, in particular greater than or equal to 0.35 and/or less than or equal to 0.70, in particular less than or equal to 0.60, in particular less than or equal to 0.55.

In some embodiments, the polyester of the first thermoplastic material and polyester of the second thermoplastic material may have different names, for example one is based on PLA and the other on PBAT or vice versa or for example one is based on a PET, a Bio-PET, a biodegradable polyester, a PHA and/or a PLA and/or a PBAT and/or a PBS and/or a PBS A and/or a PES and/or a PTS and/or a PTAT and/or a PCL and/or a TPS, and the other based on a second thermoplastic material with a different name and also selected from this same list.

In some embodiments, the second material may comprise a non-thermoplastic material that is present at a content greater than or equal to 1.5% by mass of the total composition of the retaining device, in particular greater than or equal to 2%, in particular greater than or equal to 4% and/or less than or equal to 15%, in particular less than or equal to 12%, in particular less than or equal to 10%.

In some embodiments, the second material may comprise a mineral and/or vegetable filler whose content is less than or equal to 25% by mass of the total composition of the retaining device, in particular less than or equal to 20% and/or greater than or equal to 5%, in particular greater than or equal to 10%, in particular greater than or equal to 13%.

In some embodiments, the retaining device may have a mass less than or equal to 200 g/m², in particular less than or equal to 150 g/cm², in particular less than or equal to 120 g/cm².

In some embodiments, the retaining elements may have a height greater than or equal to 120 μm and/or less than or equal to 400 μm.

In some embodiments, the base may have a thickness greater than or equal to 25 μm, in particular greater than or equal to 40 μm and/or less than or equal to 150 μm, in particular less than or equal to 100 μm.

In some embodiments, the rod of the retaining elements has a first dimension along the direction MD and a second dimension along the direction CD, the ratio of the first dimension to the second dimension is greater than 0.7, in particular greater than 0.8, in particular greater than 0.9 and/or less than 1.3, in particular less than 1.2, in particular less than 1.1. Thus, with such a ratio for the rod of the retaining elements, the effect of the invention is even more expressed, in particular with regard to the grip.

The thickness of the base is measured between the upper face and the lower face in a direction perpendicular to the upper face and the lower face. The upper face of the base may be flat or substantially flat. The lower face of the base may be flat or substantially flat.

In some embodiments, each retaining element may comprise a rod surmounted by a head; each rod comprising a lower end connected to the base, and an opposite upper end from which the head extends.

The head typically extends from the upper end of the rod. The head may have at least one portion extending beyond the upper end of the rod, in order to define a gripping portion or lobe adapted to engage fibers and/or loops and/or additional retaining elements to produce a self-gripping connection. The head thus typically has a maximum section having a surface strictly greater than the surface of the upper end of the rod.

In some embodiments, the head has a thickness greater than or equal to 20 μm, in particular greater than or equal to 40 μm, and/or less than or equal to 150 μm, in particular less than or equal to 70 μm, more particularly less than or equal to 60 μm.

In some embodiments, the head has a thickness greater than or equal to 120 μm, in particular greater than or equal to 150 μm, and/or less than or equal to 550 μm, in particular less than or equal to 500 μm, more particularly less than or equal to 480 μm.

By way of non-limiting example, in a first direction, for example in the direction MD (according to the acronym for “Machine Direction”, the rod has a minimum width and the head has a maximum width, the ratio of the minimum width of the rod to the maximum width of the head is greater than or equal to 0.5, in particular greater than or equal to 0.55, in particular greater than or equal to 0.6 and/or less than or equal to 1.0, in particular less than or equal to 0.95, in particular less than or equal to 0.90.

By way of non-limiting example, in a second direction, in particular perpendicular to the first direction, for example in the direction CD (according to the acronym for “Cross Direction”), the rod has a minimum width and the head has a maximum width, the ratio of the minimum width of the rod to the maximum width of the head is greater than or equal to 0.35, in particular greater than or equal to 0.4, in particular greater than or equal to 0.45 and/or less than or equal to 1.0, in particular less than or equal to 0.8, in particular less than or equal to 0.70, in particular less than or equal to 0.65.

By way of non-limiting example, in a second direction, in particular perpendicular to the first direction, the rod has a minimum width and the head has a maximum width, the ratio of the minimum width of the rod to the maximum width of the head is greater than or equal to 0.90, in particular greater than or equal to 0.95, and/or less than or equal to 1.1, in particular less than or equal to 1.05.

In some embodiments, the rod has a height greater than or equal to 150 μm, in particular greater than or equal to 200 μm and/or less than or equal to 330 μm, in particular less than or equal to 300 μm.

In some embodiments, the ratio of the thickness of the head to the height of the rod is greater than or equal to 0.05, in particular greater than or equal to 0.1, and/or less than or equal to 0.7, in particular less than or equal to 0.3, in some cases less than or equal to 0.25.

In some embodiments, in a first direction, for example in the direction MD, the rod having a proximal portion arranged on the base side and a distal portion arranged on the head side, the width of the rod at its proximal portion may be greater than the width of the rod at its distal portion.

In some embodiments, in a second direction, in particular perpendicular to the first direction, for example in the direction CD, the rod having a proximal portion arranged on the base side and a distal portion arranged on the head side, the width of the rod at its proximal portion may be greater than the width of the rod at its distal portion.

In some embodiments, the retaining element may have at least one plane of symmetry perpendicular to the plane formed by the base and passing through the center of the rod and/or the center of the head.

In some embodiments, the retaining element may have a single plane of symmetry perpendicular to the plane formed by the base and passing through the center of the rod and/or the center of the head.

In some embodiments, the retaining element may have at least two planes of symmetry perpendicular to the plane formed by the base and passing through the center of the rod and/or the center of the head, in particular the two planes of symmetry of the retaining element being perpendicular to each other.

In some embodiments, the retaining elements may typically be made from a preform (not shown) resulting from molding, in particular by continuous or discontinuous injection or by extrusion comprising at least one rod, and optionally one head, the upper end of the rod and/or the head of the preform of which is deformed or are deformed. The deformation is typically carried out by calendering, for example by hot calendering, for example in one or two calendering steps.

In some embodiments, the retaining elements may typically be formed integrally with the base, in particular, the rod and head of the retaining elements are formed integrally with the base. In some embodiments, the material composition of the retaining elements and/or the base is homogeneously distributed in the retaining elements (including the rod and/or the head) and/or the base, respectively.

In some embodiments, the first thermoplastic material may be void of or free of an elastomeric material and/or a thermoplastic elastomer, namely a material as defined in standard ISO 18064 of April 2022. In other words, the first thermoplastic material may be a non-elastomeric material and/or a non-elastomeric thermoplastic, an elastomeric material and/or an elastomeric thermoplastic meeting the standard ISO 18064 of April 2022. Thus, with such a feature, the manufacturing cost of such a device is reduced.

In some embodiments, the second thermoplastic material may be void of or free of an elastomeric material and/or a thermoplastic elastomer, namely a material as defined in standard ISO 18064 of April 2022. In other words, the second thermoplastic material may be a non-elastomeric material and/or a non-elastomeric thermoplastic, an elastomeric material and/or an elastomeric thermoplastic meeting standard ISO 18064 of April 2022. Thus, with such a feature, the manufacturing cost of such a device is reduced.

In some embodiments, the retaining device is produced by extruding material, and thus forms an extruded retaining device. Such a retaining device has a direction MD and a direction CD, transverse to the direction MD.

In some embodiments, each retaining element comprising a rod, the rods may be produced by molding, namely that the rods have a surface which is produced directly by extrusion or produced directly by extrusion and at least one calendering step for the formation (or deformation) of a head. In other words, the rods are free of a cutting step and/or a stretching step which are steps likely to weaken the product, in particular to introduce “spiral break” making the retaining device easily tearable during winding and/or unwinding. In some cases, the rods are provided with two opposite flat faces, in particular two opposite flat faces perpendicular to the direction MD and/or CD.

In some embodiments, the retaining elements and the base may comprise predominantly, in mass percentage, in particular consist of being formed, of said first thermoplastic material and of said second material.

In some embodiments, the rod and the head of the retaining elements as well as the base may comprise predominantly, in mass percentage, in particular consist of the fact that they are formed, of said first thermoplastic material and of said second material.

In some embodiments, the retaining elements may be arranged in the form of rows and columns which may be aligned, or arranged in a staggered manner. More specifically, the different retaining elements may be arranged so as to be aligned in the transverse direction CD and/or in the machine direction MD, or be offset so as to form a staggered or honeycomb pattern, two successive rows or two columns then being offset respectively in the transverse direction and/or in the machine direction, by a pitch corresponding to half the transverse interval and/or half the machine interval respectively.

In some embodiments, each retaining element may have a head thickness which is the distance measured in a direction perpendicular to the base between the upper end of the rod and the upper end of the retaining element.

In some embodiments, each retaining element may have a grip height which is the distance measured in a direction perpendicular to the base between the lower end of the head and the upper end of the head, the grip height of the head is greater than or equal to the thickness of the head.

In some embodiments, the retaining elements may typically have a hooking height greater than or equal to 30 μm, in particular greater than or equal to 40 μm and/or less than or equal to 120 μm, in particular less than or equal to 70 μm, in particular less than or equal to 60 μm.

In some embodiments, the retaining elements may typically have a hooking height greater than or equal to 150 μm, in particular greater than or equal to 200 μm and/or less than or equal to 650 μm, in particular less than or equal to 550 μm, in particular less than or equal to 500 μm, in particular less than or equal to 370 μm.

In some embodiments, the retaining elements may be typically adapted or formed such that the retaining device may cooperate with loops to achieve contact closure.

Generally speaking, it is possible to distinguish two types of self-gripping connection, those whose retaining elements cooperate with other retaining elements of the same types/natures or in the form of fibers and/or filaments and/or loops to produce mechanical connections and those whose retaining elements cooperate with surfaces to produce adhesive type connections (Gecko) or using Van der Waals forces. Although this second type of connection can be considered in certain cases as a so-called self-gripping connection, such retaining elements using Van der Waals forces have a behavior and properties that are completely different from those using mechanical connections. Indeed, in the case of retaining elements using Van der Waal forces, only the upper faces of the heads ensure the attachment to the receiving surface whereas in the case of retaining elements for mechanical attachment, the attachment is not ensured by the upper face of the heads but by the lower face of the head and the rod. According to one example, the retaining device according to the invention is only suitable for making mechanical connections.

In some embodiments, the retaining device may comprise a number of retaining elements per cm² greater than or equal to 10, in particular greater than or equal to 50, in particular greater than or equal to 125, in particular greater than or equal to 200 and/or less than or equal to 700, in particular less than or equal to 550, in particular less than or equal to 450, in certain cases less than or equal to 315.

In some embodiments, the retaining device may have a peel strength greater than or equal to 2 N, in particular greater than or equal to 3 N, measured according to the method described in the remainder of the description.

### Method for Measuring Peel Resistance.

To measure peeling performance as a function of overfeeding, a measurement of the resistance to opening at 180° of an assembled comfort hook/strip pair is used. A strip of hooks, for example, 15 mm wide and 25.4 mm long, assembled on an 80 g/m² paper support and 25.4 mm wide, is pressed onto a comfort strip sample measuring 50 mm×50 mm using a single 2 kg roll, the relative orientations of the products being identical to those used on the diaper. A pull of 1 kg is then applied for 10 seconds to the hook support in order to simulate the closure of a diaper, in particular a diaper with elastic ears. The paper supporting the hook is then inserted into the upper movable jaw of a traction frame, for example type 1/M from MTS System equipped with a 100 N load cell, and the comfort strip is inserted into the lower jaw. The distance between the two jaws is 50 mm. To measure the opening force, the upper part of the frame then performs a translation from bottom to top at a speed of 305 mm/min. The value of the maximum force supplied by the machine is then recorded, as well as, if necessary, the energy value corresponding to the area under the surface of the test curve taken over the first 13 millimeters of travel of the traction frame. When the width of the product is different from 15 mm, the value obtained is recalculated proportionally to 15 mm.

In some embodiments, the retaining device may have a shear strength greater than or equal to 35 N, in particular greater than or equal to 45 N, measured according to the method described in the remainder of the description.

### Method for Measuring Shear Strength.

To measure shear performance as a function of overfeeding, a 50 mm×50 mm sample of comfort strip is taken and glued to a rigid plate, for example metal rigid plate, with double-sided adhesive.

A strip of hooks, for example 15 mm wide and 25.4 mm long, assembled on a 250 g/m² paper support, is inserted by the operator onto the comfort strip, respecting the relative orientations of the products on the layer, and pressure is exerted for 3 seconds by the operator with the thumb.

A pull of 1 kg is then applied for 5 seconds to the hook support in order to simulate the closure of a diaper, particularly a diaper with elastic ears.

The metal plate supporting the comfort strip is inserted into the movable upper jaw, for example of a traction frame of the type 1/M from MTS System equipped with a 100 N load cell.

The paper supporting the hook is then inserted into the fixed lower jaw.

The movement of the frame will be in the same direction as the 1 kg pull. The distance between the two jaws is 76 mm. To measure the opening force, the upper part of the frame then translates from bottom to top at a constant speed of 305 mm/min. The test is carried out until the loop and hook are completely disengaged. The maximum force value is then recorded on the curve obtained. When the product width is different from 15 mm, the value obtained is recalculated proportionally to 15 mm.

This disclosure also relates to a laminated assembly comprising the retaining device as defined above and a non-woven web.

By way of non-limiting examples, the non-woven web may be produced using dry-laid, wet-laid, or spun-laid (melted/extruded process) technology and consolidated by mechanical, thermal, chemical and/or adhesive bonding.

According to one example, the web is made of a consolidated carded non-woven fabric, in particular a Spunlace non-woven fabric, that is to say consolidated by hydro-bonding.

The non-woven web may be made from various synthetic and/or natural materials. Exemplary natural materials are cellulose fibers, such as cotton, jute, linen and the like and may also include re-processed cellulose fibers, such as rayon or viscose. Natural fibers for a non-woven material may be prepared using various methods such as carding. Exemplary synthetic materials include, but without limitation, synthetic plastic polymers, which are known to form fibers that include, but without limitation, polyolefins, for example, polyethylene, polypropylene, polybutylene and the like; polyamide, for example, polyamide 6, polyamide 6.6, polyamide 10, polyamide 12 and the like; polyesters, for example polyethylene terephthalates, polybutylene terephthalates, polylactic acids and the like, polycarbonates, polystyrenes, thermoplastic elastomers, polymeric vinyls, polyurethanes and mixtures and copolymers thereof. For example, the non-woven may be a non-woven of the Spunbond, Spunmelt, carded thermally bonded, SMS, SMMS, SS, SSS, SSMMS, SSMMMS, Air through or other type. For example, the non-woven may be a non-woven comprising a different combination of Spunbond “S” and Meltblown “M” layers. These examples are given without limitation.

This disclosure also relates to a method for manufacturing a retaining device as defined above comprising the following steps:
1 the first thermoplastic material having a flexural modulus greater than or equal to 200 MPa and less than or equal to 4500 MPa and the second material are provided, the second material being selected from the group consisting of:
a second thermoplastic material having a flexural modulus different by at least 10% and/or at least 100 MPa from the flexural modulus of the first thermoplastic material,
of a non-thermoplastic material present at a content strictly greater than 1% by mass of the total composition of the retaining device,
a mineral and/or vegetable filler whose content is less than or equal to 30% by mass of the total composition of the retaining device, or
a combination thereof.
the first thermoplastic material and the second material are mixed
the retaining device is formed from the mixture of the first thermoplastic material and the second material.

In some embodiments, the step of mixing the first thermoplastic material and the second material is carried out in line, for example in the extruder and/or in a dosing device arranged at the inlet of the extruder.

In some embodiments, after the step of mixing the first thermoplastic material and the second material, the method comprises the steps of:
the mixture of the first thermoplastic material and the second material is packaged for transport, then
the mixture of the first thermoplastic material and the second material is deconditioned to form the retaining device.

In some embodiments, the step of forming the retaining device comprises a sub-step of deforming preforms of the retaining elements.

The present disclosure further relates to an absorbent article, for example of the baby diaper type or an adult incontinence diaper, comprising a top sheet, a bottom sheet and an absorbent core, arranged between the two top and bottom sheets, and at least one retaining device as defined previously.

In some embodiments, the absorbent article comprises at least complementary retaining elements, arranged and configured to cooperate with the retaining elements of the retaining device to achieve the closure of the absorbent article and/or to achieve the assembly, in particular the temporary assembly, of one or more sub-assemblies of the absorbent article.

### BRIEF DESCRIPTION OF THE DRAWINGS

Other features and advantages of the object of the present disclosure will emerge from the following description of embodiments, given as non-limiting examples, with reference to the appended figures.

FIG. 1 is a schematic sectional view of a molding apparatus for forming a retaining device.

FIG. 2 is a flowchart showing the steps in a method for manufacturing a retaining device.

FIG. 3 is a schematic perspective view of a retaining element according to a first embodiment.

FIG. 4 is a schematic perspective view of a retaining element according to a second embodiment.

### DETAILED DESCRIPTION

The invention will be illustrated by examples (Ex 1 to Ex 6) and comparative examples (Comp 1 to Comp 4).

### Example 1 (Ex 1)

Example 1 is a mixture of two polypropylenes and a mineral filler.

The first thermoplastic material is a first polypropylene with an MFR (230° C./2.16 kg) of 20 g/10 min and a flexural modulus of 2200 MPa.

The second material is a mixture of the second thermoplastic material, that is to say a second polypropylene having an MFR (230° C./2.16 kg) of 60 g/10 min and a flexural modulus of 1800 MPa, and the mineral filler, that is to say a TiO₂-based colorant.

The mass composition is as follows: 64.2% of the first polypropylene, 35% of the second polypropylene and 0.8% of the colorant.

### Example 2 (Ex 2)

Example 2 is a mixture of two polypropylenes and a mineral filler.

The first thermoplastic material is a first polypropylene with an MFR (230° C./2.16 kg) of 40 g/10 min and a flexural modulus of 1700 MPa.

The second material is a mixture of the second thermoplastic material, that is to say a second polypropylene having an MFR (230° C./2.16 kg) of 25 g/10 min and a flexural modulus of 1500 MPa, and the mineral filler, that is to say a TiO2-based colorant.

The mass composition is as follows: 65% of the first polypropylene, 34% of the second polypropylene and 1% of the colorant.

### Example 3 (Ex 3)

Example 3 is a mixture of a PLA and a plasticizer.

The first thermoplastic material is a PLA with an MFR (190° C./2.16 kg) of 30 g/10 min and a flexural modulus of 3400 MPa.

The second material is a non-thermoplastic material: a plasticizer based on a citric acid ester with a molecular mass of less than 600 g/mol. The plasticizer also has a lubricating and flow-enhancing effect.

The mass composition is as follows: between 85% and 90% PLA and between 15% and 10% plasticizer.

### Example 4 (Ex 4)

Example 4 is a mixture of a PLA and a plasticizer.

The first thermoplastic material is a PLA with an MFR (190° C./2.16 kg) of 30 g/10 min and a flexural modulus of 3400 MPa.

The second material is a non-thermoplastic material: a plasticizer based on a citric acid ester with a molecular mass of less than 600 g/mol. The plasticizer also has a lubricating and flow-enhancing effect.

The mass composition is as follows: between 80% and 85% PLA and between 20% and 15% plasticizer.

### Example 5 (Ex 5)

Example 5 is a mixture of a PLA and a plasticizer.

The first thermoplastic material is a PLA with an MFR (190° C./2.16 kg) of 35 g/10 min, a flexural modulus of 3600 MPa and a number-average molecular mass of approximately 44000 g/mol.

The second material is a mixture of a second thermoplastic material, non-thermoplastic materials and a mineral filler.

The second thermoplastic material is a mixture of PLA and PBAT with an MFR (190° C./2.16 kg) of 3-5 g/10 min and a tensile modulus of 185-420 MPa.

The non-thermoplastic material is a mixture of a lubricant and a flow enhancer each having a molecular mass less than or equal to 1100 g/mol. According to one example, the lubricant comprises a processing aid and a slip agent, the slip agent being in particular based on fatty acid derivatives, for example a fatty amide.

The mineral filler is talc.

The mass composition is as follows: 52% (+/−2%) of PLA, 23 (+/−2%) of the second thermoplastic material, around 2.5% of lubricant, around 6% of flow enhancer and around 16% of talc.

### Comparative Example 1 (Comp 1)

Comparative Example 1 consists of a polypropylene having an MFR (230° C./2.16 kg) of 35 g/10 min and a flexural modulus of 1600 MPa.

### Comparative Example 2 (Comp 2)

Comparative Example 2 consists of a polypropylene having an MFR (230° C./2.16 kg) of 100 g/10 min and a flexural modulus of 1500 MPa.

### Comparative Example 3 (Comp 3)

Comparative example 3 consists of a PLA having an MFR (190° C./2.16 kg) of 30 g/10 min and a flexural modulus of 3400 MPa.

### Example 6 (Ex 6)

Example 6 is a mixture of two polypropylenes and a mineral filler.

The first thermoplastic material is a first polypropylene having an MFR (230° C./2.16 kg) of 35 g/10 min and a flexural modulus of 1210 MPa.

The second material is a mixture of a second thermoplastic material, that is to say a second polypropylene having an MFR (230° C./2.16 kg) of 60 g/10 min and a flexural modulus of 1800 MPa, and a mineral filler, that is to say a TiO₂-based colorant.

The mass composition is as follows: 79.2% of the first polypropylene, 20% of the second polypropylene and 0.8% of the TiO₂-based colorant.

### Comparative Example 4 (Comp 4)

Comparative Example 4 consists of a polypropylene having an MFR (230° C./2.16 kg) of 35 g/10 min and a flexural modulus of 1210 MPa.

### Forming the Retaining Device

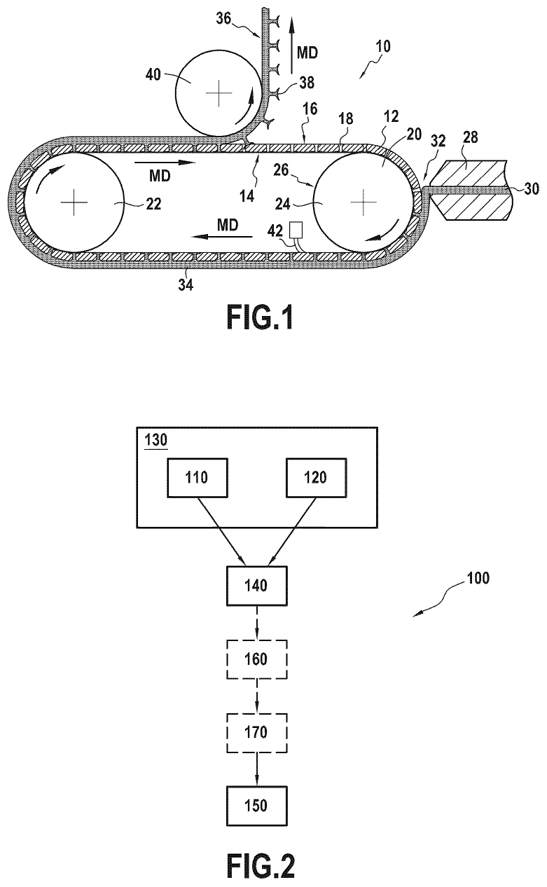

FIG. 1 shows a schematic sectional view of a molding apparatus 10 for forming a retaining device. The molding apparatus 10 comprises a molding strip 12 closed on itself and having an inner face 14, an outer face 16, a plurality of through cavities 18 extending from the outer face 16 to the inner face 14.

The molding strip 12 is stretched over means for rotating the molding strip 12, for example two rotational drive rollers 20, 22. One of the rotational drive rollers 20 of the molding strip 12 can act as a molding support 24.

The molding support 24 includes a molding face 26 intended to contact the inner face 14 of the molding strip 12. The inner face 14 of the molding strip 12 bears against the rotational drive rollers 20 of the molding strip 12.

The molding apparatus 10 also comprises a device 28 for dispensing the plastic material 30 into the cavities 18 of the molding strip 12. In FIG. 1, the device 28 for dispensing a material, for example a plastic material 30 (namely the mixture of the first thermoplastic material and the second material), is arranged on the side of the outer face 16 of the molding strip 12, opposite the molding support 26, that is to say that the plastic material 30 is dispensed into the cavities 18 of the molding strip when the inner face 14 of the molding strip 12 is bearing against the molding face 26 of the molding support 24.

For example, the dispensing device 28 may be a head for injecting plastic material. The head for injecting plastic material comprises an opening whose width in the transverse direction is less than or equal to the width in the transverse direction of the molding strip 12.

In FIG. 1, the dispensing device 28 is disposed at a certain distance from the outer face 16 of the molding strip 12 so as to form an air gap 32 between the molding strip 12 and the dispensing device 28.

When dispensing the plastic material 30 into the cavities 18 of the molding strip 12, a base 34 is also formed on the outer face 16 of the molding strip 12, so as to form, once demolded, a ribbon 36 including a base 34 on which a plurality of retaining elements 38 or a plurality of retaining element preforms is formed.

The molding apparatus 10 also includes a demolding roller 40. The demolding roller 40 may, for example, be configured to separate the base 34 of the ribbon 36 from the molding strip 12 under the effect of the tension of the ribbon 36 and its change of direction. The demolding roller 40 may be a suction roller or comprise a rubber coating.

It will be noted that the molding apparatus 10 may also include a device for removing excess plastic material, such as a scraper 42 disposed, in the example of FIG. 1, on the side of the inner face 14 of the molding strip 12 and after the molding support 24, in the direction of travel of the molding strip 12. It is therefore understood that this scraper 42 is disposed after the dispensing device 28.

The compositions of Examples 1 to 5 and Comparative Examples 1 to 3 were made and the retaining devices were formed in the molding apparatus 10.

It is understood that the molding apparatus 10 is provided by way of example. Other apparatus may be used to form retaining devices from the compositions of Examples 1-5 and Comparative Examples 1-3.

By way of non-limiting example, FIG. 2 shows the method 100 for manufacturing the retaining device. The method 100 comprises a step 130 of providing the first thermoplastic material 110 and the second material 120, a step 140 of mixing the first thermoplastic material 110 and the second material 120 and a step 150 of forming the retaining device from the mixture of the first thermoplastic material and the second material.

The mixture of the first thermoplastic material and the second material is represented in FIG. 1 by the plastic material 30 leaving the dispensing device 28.

Between the mixing step 140 and the forming step 150, the mixture of the first thermoplastic material and the second material can be conditioned 160 to transport the mixture and, once the mixture of the first thermoplastic material and the second material has arrived at its destination, the mixture of the first thermoplastic material and the second material can be unconditioned 170 and the forming step 150 can be carried out.

Alternatively, the mixing of the first thermoplastic material and the second material can be done in-line, that is to say that the step 130 of supplying the first thermoplastic material 110 and the second material 120, and the step 140 of mixing the first thermoplastic material 110 and the second material 120 is carried out in equipment directly connected to the dispensing device 28, for example.

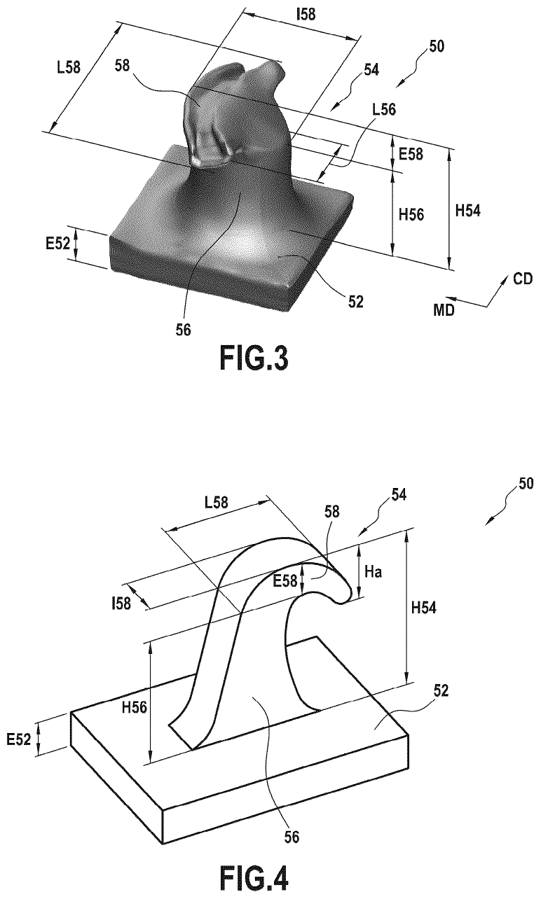

FIGS. 3 and 4 are partial perspective views of two retaining devices 50.

In FIGS. 3 and 4, common elements are identified by identical numerical references.

Also shown, as a non-limiting example, are the directions MD and CD.

The retaining device 50 comprises a base 52 having an upper face and a lower face and a plurality of retaining elements 54 extending from the upper face of the base 52. In FIGS. 3 and 4, a single retaining element 54 has been shown.

The retaining element comprises a rod 56, extending from the upper face of the base 52, surmounted by a head 58. The rod comprises a lower end connected to the base 52, and an opposite upper end from which the head 58 extends.

The head 58 may have at least one portion extending beyond the upper end of the rod, in order to define a gripping portion or lobe adapted to engage fibers and/or loops and/or complementary retaining elements to produce a self-gripping connection. The head 58 thus typically has a maximum section having a surface strictly greater than the surface of the upper end of the rod 56.

The thicknesses and heights are measured in a direction perpendicular to the upper face and/or the lower face of the base, for example perpendicular to the plane formed by the directions MD and CD and the widths are measured in a plane parallel to the upper face and/or the lower face, for example in a plane parallel to the plane formed by the directions MD and CD.

The base 52 has a thickness E52 measured between the upper face and lower face of the base 52; the retaining element 54 has a height H54; the rod 56 has a height H56 and a width L56; the head has a thickness E58 and two widths L58 and I58. The width L58 is the maximum width of the head 58.

The hooking height Ha has been represented in FIG. 4.

Table 1 presents the results of peel strength and shear strength measurements for hook-type retaining devices. The peel strength and shear strength values are reported for retaining devices with a width of 15 mm.

TABLE 1

| | Shaping of | | |
|---|---|---|---|
| | preforms | | |
| | and/or | | |
| | retaining | Peel strength | Shear strength |
| | elements | (N) | (N) |
| Ex 1 | Yes | 5.1 +/− 0.7 | 72 +/− 7 |
| Ex 2 | Yes | 3.7 +/− 0.6 | Not available |
| Ex 3 | Yes | 2.8 +/− 1.2 | 60 +/− 16 |
| Ex 4 | Yes | 2.1 +/− 1.0 | 47 +/− 16 |
| Ex 5 | Yes | 2.2 +/− 1.0 | 63 +/− 9 |
| Ex 6 | Yes | 4.3 +/− 0.7 | 66.8 +/− 9 |
| Comp 1 | Yes | 3.5 +/− 0.5 | 68 +/− 4 |
| Comp 2 | Yes | 3.9 +/− 0.7 | 63 +/− 6 |
| Comp 3 | No | — | — |
| Comp 4 | Yes | 3.4 +/− 0.7 | 63 +/− 6 |

The peel strength and shear strength values for Comparative Example 3 could not be measured. This is because this material, injection-grade PLA alone, cannot be molded. The molding of the retaining device is partial, so that the molding cavities are not completely filled because the material is too viscous. In this case, increasing the forming temperature to fluidize the material is not possible due to the thermal sensitivity of compostable materials. In addition, with a high flexural modulus, demolding the retaining elements and/or preforms is very difficult, leading to their breakage or excessive deformation of the rods. It is therefore not possible to obtain satisfactory retaining elements and/or preforms, particularly for the production of retaining elements with heights of less than 350 microns and/or a base with a thickness of less than 120 micrometers, particularly less than 100 micrometers.

The peel strength and shear strength values for Comparative Example 4 could not be measured. This is because this material, comprising polypropylene alone, cannot be molded. The molding of the retaining device is partial such that the molding cavities are not completely filled because the material is too viscous. It is therefore not possible to obtain satisfactory retaining elements and/or preforms, in particular for the production of retaining elements having heights of less than 350 microns and/or a base having a thickness of less than 120 micrometers, in particular less than 100 micrometers.

The values of Examples 1, 2 and 6 are to be compared with the values of Comparative Examples 1, 2 and 4. The values of Examples 3-5 are to be compared with the values of Comparative Example 3. More specifically, the values of Example 1 are to be compared with the values of Comparative Example 1. The values of Example 6 are to be compared with the values of Comparative Example 4.

For Examples 1 and 2 and Comparative Examples 1 and 2, the peel strength and shear strength measurements were carried out on retaining devices having a width of 15 mm.

For Example 3, the peel strength and shear strength measurements were carried out on retaining devices with a width of 19 mm. The peel strength value is 3.6+/−1.5 N, or a value of 2.8+/−1.2 N relative to a width of 15 mm, and the shear strength value is 76+/−20 N, or a value of 60+/−16 N relative to a width of 15 mm.

For Example 4, the peel strength and shear strength measurements were carried out on retaining devices with a width of 19 mm. The peel strength value is 2.6+/−1.2 N, or a value of 2.1+/−1.0 N relative to a width of 15 mm, and the shear strength value is 60+/−20 N, or a value of 47+/−16 N relative to a width of 15 mm.

For Example 5, the peel strength and shear strength measurements were carried out on retaining devices with a width of 22 mm. The peel strength value is 3.2+/−1.4 N, or a value of 2.2+/−1.0 N relative to a width of 15 mm, and the shear strength value is 93+/−13 N, or a value of 63+/−9 N relative to a width of 15 mm.

As can be seen, the peel strength values of Examples 1, 2 and 6 are similar to, or even higher than, the values of Comparative Examples 1, 2 and 4. The same is true for the shear strength values.

It should be noted that Examples 3 to 5 should be compared with Comparative Example 3. However, the peel strength values of Examples 3 to 5, although lower than the values of Comparative Examples 1 and 2, are satisfactory, particularly for the intended application. The shear strength values are lower than, or even similar to, the values of Comparative Examples 1 and 2.

Examples 1 to 6 allow to obtain satisfactory retaining devices which are compostable in an industrial environment, for example according to standard EN 13432:2000, or which are based on compostable materials, and/or derived from biomass, for example according to standard EN 16785-1:2016 and/or standard ASTM D 6866:2022, and/or which can always be taken into account at the end of their life in industrial composting and/or recycling channels.

By comparing example 6 with comparative example 4, it can be noted that the average performances (average peeling and/or shear) of the retaining elements are improved while providing an equally improved economic interest and above all by bypassing a source of supply of a single, demanding and very specific material, and by using a wide capacity (or wide range) of use (selection) of a first less specific or demanding material and a second less specific or demanding material as defined above.

Although the present disclosure has been described with reference to a specific exemplary embodiment, it is obvious that various modifications and changes may be made to these examples without departing from the general scope of the invention as defined by the claims. Furthermore, individual features of the various embodiments mentioned may be combined in additional embodiments. Therefore, the description and drawings are to be considered in an illustrative rather than restrictive sense.

1. A retaining device, comprising:
a base extending in a longitudinal direction having an upper face and a lower face,
a plurality of retaining elements extending from the upper face of the base, each retaining element comprising a rod,
the retaining device comprising predominantly, in mass percentage, in particular consisting in that it is formed from a first thermoplastic material having a flexural modulus, measured according to standard ISO 178:2019, greater than or equal to 200 MPa and less than or equal to 4500 MPa and a second material, the second material being chosen from the group consisting of:
a second thermoplastic material having a flexural modulus different by at least 10% and/or at least 100 MPa from the flexural modulus of the first thermoplastic material,
of a non-thermoplastic material present at a content strictly greater than 1% by mass of the total composition of the retaining device,
a vegetable filler whose content is less than or equal to 30% by mass of the total composition of the retaining device, or
a combination thereof.

2. The retaining device according to claim 1, wherein the first thermoplastic material has a flexural modulus greater than or equal to 800 MPa and less than or equal to 4000 MPa.

3. The retaining device according to claim 1, wherein the first thermoplastic material has a number-average molecular mass greater than or equal to 20000 g/mol.

4. The retaining device according to claim 3, wherein the second material comprises a non-thermoplastic material having a molecular mass at least 10% lower than the number-average molecular mass of the first thermoplastic material and/or less than or equal to 15000 g/mol.

5. The retaining device according to claim 1, wherein the first thermoplastic material has an MFR greater than or equal to 1 g/10 min.

6. The retaining device according to claim 5, wherein the second material, in particular the second thermoplastic material, has an MFR different by at least 10% and/or at least 5 g/10 min from the MFR of the first thermoplastic material.

7. The retaining device according to claim 1, wherein the non-thermoplastic material has a molecular mass greater than or equal to 250 g/mol and/or less than or equal to 19 000 g/mol.

8. The retaining device according to claim 7, wherein the first thermoplastic material is based on a polyester, and the second material comprises a non-thermoplastic material present at a content greater than or equal to 1.5% by mass of the total composition of the retaining device, and/or less than or equal to 20%.

9. The retaining device according to claim 1, wherein the ratio of the mass percentage of the second material to the first thermoplastic material is greater than or equal to 0.02 and/or less than or equal to 0.30.

10. The retaining device according to claim 1, wherein the first thermoplastic material is a first polyolefin-based material, and the second material comprises the second thermoplastic material, the second thermoplastic material being a second polyolefin-based material.

11. The retaining device according to claim 10, wherein the ratio of the mass percentage of the second thermoplastic material to the first thermoplastic material is greater than or equal to 0.3 and/or less than or equal to 0.7.

12. The retaining device according to claim 1, wherein the first thermoplastic material is based on a first polyester, and the second material comprises the second thermoplastic material, the second thermoplastic material being based on a second polyester.

13. The retaining device according to claim 12, the ratio of the mass percentage of the second thermoplastic material to the first thermoplastic material is greater than or equal to 0.20 and/or less than or equal to 0.70.

14. The retaining device according to claim 1, wherein each retaining element comprises a rod surmounted by a head, in a first direction, the rod having a proximal portion arranged on the base side and a distal portion arranged on the head side, the width of the rod at its proximal portion may be greater than the width of the rod at its distal portion.

15. The retaining device according to claim 1, wherein the second material comprises a mineral filler.

16. The retaining device according to claim 1, wherein the non-thermoplastic material is present at a content strictly greater than 1.1%.

17. The retaining device according to claim 1, wherein the non-thermoplastic material is present at a content strictly greater than 1.2%.

18. The retaining device according to claim 10, wherein the first thermoplastic material is a first material based on a first polypropylene

19. The retaining device according to claim 10, wherein the second thermoplastic material is a second polypropylene.
